(19) United States
(12) Patent Application Publication
(10) Pub. No.: US 2025/0385787 A1
(43) Pub. Date: Dec. 18, 2025

(54) CONTROLLING ACCESS TO A MEDICAL DEVICE

(71) Applicant: Loewenstein Medical Technology S.A., Luxembourg (LU)

(72) Inventor: Milos DORDEVIC, Hamburg (DE)

(21) Appl. No.: 19/236,204

(22) Filed: Jun. 12, 2025

(30) Foreign Application Priority Data
Jun. 17, 2024 (DE) 10 2024 116 911.7

(51) Int. Cl. H04L 9/08; A61M 16/00; H04L 9/32

(52) U.S. Cl. CPC H04L 9/0863; H04L 9/3226; A61M 16/024

(57) ABSTRACT

A method for granting user access to a medical device via a user device comprises the following steps, which are carried out by a processor of the medical device: generating an encrypted password by encrypting a password to be encrypted; sending the encrypted password to the user device in order to allow decryption of the encrypted password; receiving an access request which is sent by the user device) in order to request the user access, the access request comprising a decrypted password; verifying whether the decrypted password matches the password to be encrypted; if the decrypted password matches the password to be encrypted: granting the user access.

### CROSS-REFERENCE TO RELATED APPLICATIONS

The present application claims priority under 35 U.S.C. § 119 of German Patent Application No. 10 2024 116 911.7, filed Jun. 17, 2024, the entire disclosure of which is expressly incorporated by reference herein.

### BACKGROUND OF THE INVENTION

### 1. Field of the Invention

The invention relates to a method for granting user access to a medical device via a user device. The invention furthermore relates to a method for requesting user access to a medical device via a user device, to a method for operating a decryption device and to a method for controlling user access to a medical device via a user device. The invention moreover relates to an apparatus for data processing, to a computer program and to a computer-readable medium for carrying out at least one of these methods.

### 2. Discussion of Background Information

The operating system of a modern medical device, for example a ventilator, can generally be accessed via an external user device, for example a PC or a laptop, for example in order to change a hardware and/or software configuration of the medical device and/or to test particular functions of the medical device. In order to protect the medical device from unauthorized users, the access may be restricted with a password specific to the device type. Changing such a password, for example in the event of a password theft, may involve a certain expense, particularly when a large number of medical devices are affected.

In view of the foregoing it would be advantageous to have available a method that allows improved control of user access to a medical device via a user device. It further would be advantageous to have available a corresponding apparatus for data processing, a corresponding computer program and a corresponding computer-readable medium.

### SUMMARY OF THE INVENTION

In a first aspect, the invention provides a method for granting user access to a medical device via a user device. The method is carried out by a processor of the medical device and comprises: generating an encrypted password by encrypting a password to be encrypted (for example from a memory of the medical device); sending the encrypted password to the user device in order to allow (external) decryption of the encrypted password; (subsequently:) receiving an access request which is sent by the user device in order to request the user access, the access request comprising a decrypted password; verifying whether the decrypted password matches the password to be encrypted; if the decrypted password matches the password to be encrypted: granting the user access to the medical device via the user device.

Because the password can be provided only in encrypted form by the medical device and first needs to be decrypted by an external device, the risk of unintended accesses to the medical device can be considerably reduced in comparison to an embodiment without such encryption and decryption—for example an embodiment in which the medical device is protected with a password specific to the device type from a central password database.

The medical device may in general be a medical product having a processor or a computer, for example in the form of an embedded system. For example, the medical device may be a ventilator for the invasive and/or noninvasive ventilation of a patient, a cough device for assisting a patient when coughing, a monitor for monitoring a patient's vital signs, a defibrillator, a cardiac pacemaker, a hearing aid, a device for imaging diagnosis or an operation robot.

A “user device” may be understood above and below as an external computer—for example a PC, a server, a laptop, a tablet or a smartphone—for data communication with the medical device and with a decryption device, as is described below. Accordingly, the user device may comprise at least one of the following components in addition to the processor: a memory, a bus system for data communication between the memory and the processor, a data communication interface for wireless and/or wired data communication with peripheral devices (for example via the Internet).

In a second aspect, the invention provides a method for requesting user access to a medical device via a user device. The method is carried out by a processor of the user device and comprises: receiving an encrypted password sent by the medical device; generating a password request, which comprises the encrypted password, for requesting a decrypted password; sending the password request to a decryption device for decrypting the encrypted password; (subsequently:) receiving a decrypted password sent by the decryption device; generating an access request, which comprises the decrypted password, for requesting the user access; sending the access request to the medical device.

In a third aspect, the invention provides a method for operating a decryption device. The method is carried out by a processor of the decryption device and comprises: receiving a password request, which is sent by a user device, for requesting a decrypted password, the password request comprising an encrypted password, the encrypted password having been generated by a processor of a medical device by encrypting a password to be encrypted; generating a decrypted password by decrypting the encrypted password; sending the decrypted password to the user device.

A “decryption device” may be understood above and below as a further external computer—for example a further PC, a further server, a further laptop, a further tablet or a further smartphone—for data communication with the user device. Accordingly, the decryption device may comprise at least one of the following components in addition to the processor: a memory, a bus system for data communication between the memory and the processor, a data communication interface for wireless and/or wired data communication with peripheral devices (for example via the Internet). The decryption device may be secured particularly well at the hardware and/or software level against unintended accesses.

In a fourth aspect, the invention provides a method for controlling user access to a medical device via a user device. The method comprises the steps of the method described above and below according to the first aspect of the invention and the steps of the method described above and below according to the second aspect of the invention. The method may in addition comprise the steps of the method described above and below according to the third aspect of the invention.

In a fifth aspect, the invention provides an apparatus for data processing. The apparatus comprises at least one of the following components:
a first processor for a medical device, the first processor being configured to carry out the method described above and below according to the first aspect of the invention;
a second processor for a user device, the second processor being configured to carry out the method described above and below according to the second aspect of the invention;
a third processor for a decryption device, the third processor being configured to carry out the method described above and below according to the third aspect of the invention.

In addition, the apparatus may comprise at least one of the following components: a memory, a bus system for data communication between the memory and the processor in question, a data communication interface for wireless and/or wired data communication with peripheral devices (for example via the Internet).

Depending on the embodiment, the apparatus may be an individual computer or a combination of a plurality of individual computers (for example in a computer network).

It is pointed out that features of the methods described above and below may also be features of the apparatus (and vice versa).

Further aspects of the invention relate to a computer program and to a computer-readable medium, on which the computer program is stored.

The computer program comprises at least one of the following instruction sets:
a first set of instructions, which cause the apparatus described above and below (for example its first processor) when the computer program is run by the apparatus to carry out the method described above and below according to the first aspect of the invention;
a second set of instructions, which cause the apparatus described above and below (for example its second processor) when the computer program is run by the apparatus to carry out the method described above and below according to the second aspect of the invention;
a third set of instructions, which cause the apparatus described above and below (for example its third processor) when the computer program is run by the apparatus to carry out the method described above and below according to the third aspect of the invention.

The computer-readable medium may be a volatile or nonvolatile data memory. For example, the computer-readable medium may be a hard disk drive, a USB storage device (universal serial bus), a RAM (random-access memory), a ROM (read-only memory), an EPROM (erasable programmable read-only memory), an EEPROM (electrically erasable programmable read-only memory), a flash memory or a combination of at least two of these examples. The computer-readable medium may also be a data communication network that makes it possible to download program code (for example via the Internet), or a cloud.

It is pointed out that features of the methods described above and below may also be features of the computer program and/or of the computer-readable medium (and vice versa).

Various embodiments of the invention are described below. These embodiments are not to be understood as a restriction of the scope of the invention.

According to one embodiment, the encrypted password may be generated by using a public key, which is stored in a memory of the medical device. The public key may form a key pair with a private key for generating the decrypted password (by decrypting an encrypted password). The encrypted password may be decryptable exclusively with the aid of the associated private key.

The key pair may be suitable for use in a symmetric and/or asymmetric encryption method. It is possible that the key pair is generated on a computer, for example the decryption device, which is separate from the medical device. The public key may then be written to a memory of the medical device, for example during the production of the medical device. On the other hand, the private key may be stored in an external memory outside the medical device and/or outside the user device, for example in a memory of the decryption device. In such an external memory, the private key may in addition be kept secret in a suitable way, i.e. protected from access by unauthorized persons. In other words, it is possible that the private key is not exchanged in any way either with the medical device or with the user device (or with another external device). For example, the same public key may be stored in the memory of various medical devices, in which case each medical device may comprise a processor for carrying out the method described above and below according to the first aspect of the invention. Each medical device therefore generates its own password, which is known only to the device in question and is provided only in encrypted form by the device in question.

According to one embodiment, the encrypted password may be generated in an asymmetric or hybrid (i.e. both symmetric and asymmetric) encryption method by using the public key. The asymmetric encryption method, which may also be referred to as a public-key method, may for example be an algorithm based on integer factorization, for example the RSA algorithm (RSA=Rivest-Shamir-Adleman), an algorithm based on the discrete logarithm problem and/or on the Diffie-Hellman problem, for example the Elgamal algorithm, an algorithm based on elliptic curves or a combination of at least two of these algorithms. Alternatively, hash-based, code-based, lattice-based or multivariate quadratic asymmetric algorithms are also possible. Such algorithms cannot be attacked, or can be attacked only with great difficulty, even with a quantum computer, and may therefore also be referred to as post-quantum algorithms. This allows particularly secure encryption of the password. The risk of unintended accesses to the medical device may therefore be minimized.

Generation of the encrypted password is also possible in a symmetric encryption method, for example with a DES algorithm (DES=Data Encryption Standard), in particular a 3DES algorithm, and/or an AES algorithm (AES=Advanced Encryption Standard). The 3DES algorithm, i.e. triple encryption with DES, also referred to as Triple DES, is particularly secure in comparison to single encryption with DES, without the key length increasing excessively.

According to one embodiment, the password (for example unencrypted) to be encrypted may further be generated by the processor of the medical device. For this purpose, a particular character string, for example random character string, may be generated and be stored as the password to be encrypted in a memory of the medical device. Expensive password management in a central database may thereby be obviated. Such decentral provision of the password may also significantly reduce the risk of password theft in comparison to central provision (for example with the aid of a password database) because the password is known only to the medical device in question and is provided only in encrypted form by the latter.

According to one embodiment, a timer may be started in response to the generation of the password to be encrypted. When the timer has elapsed, it may be determined that the password to be encrypted is invalid in its current version, so that user access to the medical device via the user device is no longer possible based on the current version. The risk of unintended access may thereby be reduced significantly in comparison to an embodiment with an unlimitedly valid password. The timer may be configured so that it elapses after a predefined duration of for example at most one hour, at most one day, at most one week, at most one month or at most one year.

According to one embodiment—when the timer has elapsed—a new version of the password to be encrypted may be generated, which is different to the current version and is then valid instead of the current version. For example, the current version may be overwritten with the new version. This allows automatic refreshing of the password to be encrypted at regular intervals. No additional—sometimes very expensive—security measures therefore need to be taken in the event of a password theft. Such refreshing of the password to be encrypted may, for example, also take place whenever the medical device is switched off or switched to standby operation.

According to one embodiment, the timer may be reset and started again in response to the generation of the new version. Accordingly, it may be determined that the new version is invalid when the timer has elapsed after being started again, so that user access to the medical device via the user device is no longer possible based on the new version. In other words, it is possible that each newly generated password is valid only for a particular duration. The risk of unintended access may be further reduced in this way.

According to one embodiment, the password to be encrypted may be newly generated each time the medical device is switched on or off or switches between different operating modes for the operation of the medical device, for example standby operation and normal (main) operation. This allows regular refreshing of the password without using a timer.

According to one embodiment, the password to be encrypted may be generated by using a random generator for generating a random character string. It is thereby possible to ensure that there is no significant relation between different versions of the password to be encrypted. For example, the random character string may be stored as the password to be encrypted in a memory of the medical device. Alternatively, the password to be encrypted may comprise a nonrandom, predetermined character string in addition to the random character string.

According to one embodiment, the access request may further comprise a user ID which is uniquely assigned to a user of the user device. For example, the user ID may comprise a digital signature for uniquely identifying the user in relation to the medical device and/or may define particular access rights of the user. With the aid of the user ID, it is possible to verify whether the user has access authorization. Accordingly, it is possible that the access request is processed further, or the user access to the medical device is granted, only if the user has access authorization.

It is also conceivable that such a user ID is received in the medical device, for example from the user device and/or from a mobile data carrier, for example a USB stick, before the encrypted password is sent to the user device. Accordingly, it is possible that the encrypted password is sent to the user device only if it is established with the aid of the user ID that the user has access authorization.

According to one embodiment, the password request may further comprise a user ID which is uniquely assigned to a user of the user device. For example, the user ID may comprise a digital signature for uniquely identifying the user in relation to the decryption device and/or may define particular access rights of the user. With the aid of the user ID, it is possible to verify whether the user has access authorization. Accordingly it is possible that the password request is processed further, or the decrypted password is generated and/or sent to the user device, only if the user has access authorization.

According to one embodiment, the decrypted password may be generated by using a private key, which is stored in a memory of the decryption device. The private key may form a key pair with a public key for generating the encrypted password—for example in an asymmetric or hybrid encryption method (see further above).

### BRIEF DESCRIPTION OF THE DRAWINGS

Embodiments of the invention are described below with reference to the appended drawings. Neither the description nor the drawings are to be understood as a restriction of the scope of the invention.

FIG. 1 shows an apparatus for data processing according to one embodiment of the invention.

FIG. 2 shows a flowchart to illustrate a method according to one embodiment of the invention.

The figures are purely schematic and not to scale. When the same reference signs are used in various drawings, these reference signs denote features that are identical or have the same effect.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

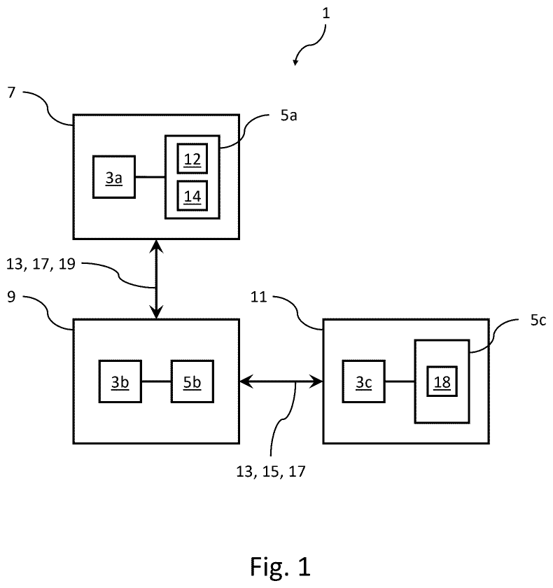

FIG. 1 shows by way of example an apparatus 1 for data processing, which comprises a first processor 3a, a second processor 3b, a third processor 3c, a first memory 5a, which is connected to the first processor 3a, a second memory 5b, which is connected to the second processor 3b, and a third memory 5c, which is connected to the third processor 3c.

The first processor 3a and the first memory 5a are components of a medical device 7, in this case a ventilator 7 for the invasive and/or non-invasive, for example pressure-controlled and/or flow-controlled and/or volume-controlled ventilation of a patient.

The second processor 3b and the second memory 5b are components of a user device 9, for example a PC, a server, a laptop, a tablet or a smartphone.

The third processor 3c and the third memory 5c are components of a decryption device 11, for example a further PC, a further server, a further laptop, a further tablet or a further smartphone.

The user device 9 may be connected to the ventilator 7 and to the decryption device 11 respectively by a wireless and/or wired data communication connection and/or via the Internet for data communication. Depending on the data communication protocol used, the data communication between the user device 9 and the ventilator 7 and/or between the user device 9 and the decryption device 11 may in addition be encrypted.

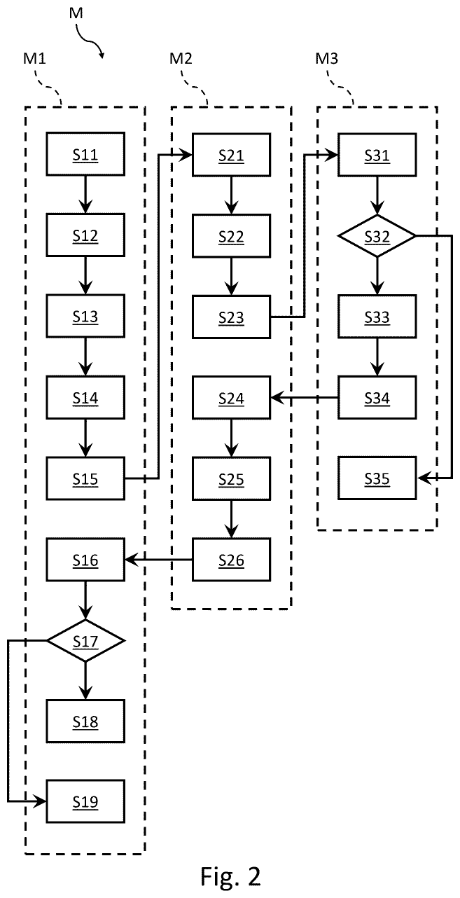

The apparatus 1 may be configured to carry out a method M (see FIG. 2) for controlling user access to the ventilator 7 via the user device 9 by running a corresponding computer program.

In this example, the method M comprises steps S11 to S19 of a first method M1 for granting the user access, steps S21 to S26 of a second method M2 for requesting the user access and steps S31 to S35 of a third method M3 for operating the decryption device 11.

The first processor 3a may be configured to carry out the first method M1 by executing a first set of instructions of the computer program, this first set of instructions being stored in the first memory 5a. Correspondingly, the second processor 3b may be configured to carry out the second method M2 by executing a second set of instructions of the computer program, this second set of instructions being stored in the second memory 5b, and the third processor 3c may be configured to carry out the third method M3 by executing a third set of instructions of the computer program, this third set of instructions being stored in the third memory 5c.

An example of a possible sequence of the method M is described below.

In step S11 a password 12 (for example unencrypted) to be encrypted is generated by the first processor 3a. For this purpose, for example, a random character string can be generated with a random generator and stored as the password 12 to be encrypted in the first memory 5a.

In step S12 a timer is started in response to the generation of the password 12 to be encrypted in step S11.

When the timer has elapsed, for example after 1, 2, 5 or 10 days, it is determined in step S13 that a current version of the password 12 to be encrypted is invalid. A new version of the password 12 to be encrypted may thereupon be automatically generated, which is then valid instead of the current version. For example, the current version may be overwritten with the new version.

When the new version is generated, the timer may for example be reset and started again, the new version remaining valid until the timer has elapsed once more. This may be repeated continuously with each newly generated password, in order to allow automatic refreshing of the password 12 to be encrypted at regular intervals.

In addition or alternatively, the password 12 to be encrypted may be newly generated each time the ventilator 7 is switched on or off or switches between standby operation and normal operation.

In step S14 the first processor 3a generates an encrypted password 13 by encrypting the password 12 to be encrypted. The encryption may, for example, take place in an asymmetric or hybrid encryption method by using a public key 14. The public key 14 may be stored in the first memory 5a.

In step S15 the encrypted password 13 is sent to the user device 9, for example via a UART interface or USB interface.

In step S21 the encrypted password 13 is received in the user device 9.

In step S22 the second processor 3b generates a password request 15, which comprises the encrypted password 13.

In step S23 the password request 15 is sent to the decryption device 11, for example via the Internet and/or a local network.

In step S31 the password request 15 is received in the decryption device 11.

It is possible that the password request 15 comprises a user ID, which is uniquely assigned to a respective user of the user device 9, in addition to the encrypted password 13. In this case, whether the user has access authorization may be verified in step S32 with the aid of the user ID.

Only if it is established in step S32 that the user has access authorization does the third processor 3c generate a decrypted password 17 in step S33 by decrypting the encrypted password 13. Otherwise, the decryption is denied in step S35. For example, the user may have access authorization if the decryption device 11 has been able to authenticate them successfully with the aid of the user ID.

It is possible that the decrypted password 17 is generated in step S33 by using a private key 18, which is stored in the third memory 5c. The private key 18 may form a key pair with the public key 14 for generating the encrypted password 13 in step S14.

In step S34 the decrypted password 17 is sent to the user device 9, for example likewise via the Internet and/or the local network.

In step S24 the decrypted password 17 is received in the user device 9.

In step S25 the second processor 3b generates an access request 19, which comprises the decrypted password 17.

In step S26 the access request 19 is sent to the ventilator 7, for example via the Internet and/or a local network.

In step S16 the access request 19 is received in the ventilator 7.

In step S17 whether the decrypted password 17 matches the password 12 to be encrypted, i.e. whether the decrypted password 17 is correct, is verified.

Only if the decrypted password 17 is correct is the user access to the ventilator 7—for example a root access to its operating system—granted in step S18, so that the user in question can change, for example, a hardware and/or software configuration of the ventilator 7, and/or test particular functions of the ventilator 7, from the user device 9. Otherwise, the user access is denied in step S19.

It is possible that the access request 19 comprises a user ID, which is uniquely assigned to the respective user, in addition to the decrypted password 17. In this case, whether the user has access authorization may be verified in step S17 with the aid of the user ID. Accordingly, the user access is granted in step S18 only on the condition that the user has access authorization. The user may have access authorization for example if the ventilator 7 has been able to authenticate them successfully with the aid of the user ID.

Finally, it is pointed out that terms such as “have”, “comprise”, “include”, “with”, etc. do not exclude other elements or steps and that indefinite articles such as “a” or “an” do not exclude the plural.

Further, it is pointed out that features or steps that are described with reference to one of the above embodiments may also be used in combination with features or steps that are described with reference to other ones of the above embodiments.

To sum up, the present invention provides the following items:

1. A method (M1) for granting user access to a medical device (7) via a user device (9), wherein the method (M1) is carried out by a processor (3a) of the medical device (7) and comprises:
generating (S14) an encrypted password (13) by encrypting a password (12) to be encrypted;
sending (S15) the encrypted password (13) to the user device (9) in order to allow decryption of the encrypted password (13);
receiving (S16) an access request (19) which is sent by the user device (9) in order to request the user access, the access request (19) comprising a decrypted password (17);
verifying (S17) whether the decrypted password (17) matches the password (12) to be encrypted;
if the decrypted password (17) matches the password (12) to be encrypted: granting (S18) the user access.

2. The method (M1) of item 1, wherein the encrypted password (13) is generated in an asymmetric or hybrid encryption method by using a public key (14), which is stored in a memory (5a) of the medical device (7), the public key (14) forming a key pair with a private key (18) for generating (S33) the decrypted password (17).

3. The method (M1) of item 1 or item 2, further comprising: generating (S11) the password (12) to be encrypted.

4. The method (M1) of item 3, further comprising:
starting (S12) a timer in response to the generation (S11) of the password (12) to be encrypted;
when the timer has elapsed:
generating (S13) a new version of the password (12) to be encrypted;
overwriting (S13) a current version of the password (12) to be encrypted with the new version.

5. The method (M1) of claim 3 or item 4,
wherein the password (12) to be encrypted is newly generated each time the medical device (7) is switched on or off or switches between different operating modes for the operation of the medical device (7), in particular standby operation and normal operation; and/or
wherein the password (12) to be encrypted is generated by using a random generator for generating a random character string.

6. The method (M1) of any one of the preceding items,
wherein the access request (19) further comprises a user ID, which is uniquely assigned to a user of the user device (9);
wherein whether the user has access authorization is verified with the aid of the user ID;
wherein the user access is granted only if the user has access authorization.

7. A method (M2) for requesting user access to a medical device (7) via a user device (9), wherein the method (M2) is carried out by a processor (3b) of the user device (9) and comprises:
receiving (S21) an encrypted password (13) sent by the medical device (7);
generating (S22) a password request (15), which comprises the encrypted password (13), for requesting a decrypted password (17);
sending (S23) the password request (15) to a decryption device (11) for decrypting the encrypted password (13);
receiving (S24) a decrypted password (17) sent by the decryption device (11);
generating (S25) an access request (19), which comprises the decrypted password (17), for requesting the user access;
sending (S26) the access request (19) to the medical device (7).

8. A method (M3) for operating a decryption device (11), wherein the method (M3) is carried out by a processor (3c) of the decryption device (11) and comprises:
receiving (S31) a password request (15), which is sent by a user device (9), for requesting a decrypted password (17), the password request (15) comprising an encrypted password (13), the encrypted password (13) having been generated by a processor (3a) of a medical device (7) by encrypting a password (12) to be encrypted;
generating (S33) the decrypted password (17) by decrypting the encrypted password (13);
sending (S34) the decrypted password (17) to the user device (9).

9. The method (M3) of item 8,
wherein the password request (15) further comprises a user ID, which is uniquely assigned to a user of the user device (9);
wherein whether the user has access authorization is verified with the aid of the user ID;
wherein the decrypted password (17) is generated and/or sent to the user device (9) only if the user has access authorization.

10. The method (M3) of item 8 or item 9,
wherein the decrypted password (17) is generated by using a private key (18), which is stored in a memory (5c) of the decryption device (11), the private key (18) forming a key pair with a public key (14) for generating (S14) the encrypted password (13) in an asymmetric or hybrid encryption method.

11. A method (M) for controlling user access to a medical device (7) via a user device (9), wherein the method (M) comprises:
the steps (S11, S12, S13, S14, S15, S16, S17, S18, S19) of the method (M1) as set forth in one of items 1 to 6;
the steps (S21, S22, S23, S24, S25, S26) of the method (M2) as set forth in claim 7.

12. The method (M) as set forth in item 11, further comprising: the steps (S31, S32, S33, S34, S35) of the method (M3) set forth in one of items 8 to 10.

13. An apparatus (1) for data processing, comprising:
a first processor (3a), which is configured to carry out the method (M1) as set forth in one of items 1 to 6; and/or
a second processor (3b), which is configured to carry out the method (M2) as set forth in item 7; and/or
a third processor (3c), which is configured to carry out the method (M3) as set forth in one of items 8 to 10.

14. A computer program, comprising:
a first set of instructions, which cause the apparatus (1) as set forth in item 13 when the computer program is run by the apparatus (1) to carry out the method (M1) as set forth in one of items 1 to 6; and/or
a second set of instructions, which cause the apparatus (1) as set forth in item 13 when the computer program is run by the apparatus (1) to carry out the method (M2) as set forth in item 7; and/or
a third set of instructions, which cause the apparatus (1) as set forth in item 13 when the computer program is run by the apparatus (1) to carry out the method (M3) as set forth in one of items 8 to 10.
15, A computer-readable medium, on which the computer program as set forth in item 14 is stored.

### LIST OF REFERENCE NUMERALS

1 apparatus for data processing
3a first processor
3b second processor
3c third processor
5a first memory
5b second memory
5c third memory
7 medical device, ventilator
9 user device
11 decryption device
12 password to be encrypted
13 encrypted password
14 public key
15 password request
17 decrypted password
18 private key
19 access request

M method for controlling user access to the medical device
M1 first method (for granting the user access)
M2 second method (for requesting the user access)
M3 third method (for operating the decryption device)
S1 . . . steps of the first method
S2 . . . steps of the second method
S3 . . . steps of the third method

What is claimed is:

1. A method (M1) for granting user access to a medical device via a user device, wherein the method (M1) is carried out by a processor of the medical device and comprises:
generating (S14) an encrypted password by encrypting a password to be encrypted;
sending (S15) the encrypted password to the user device in order to allow decryption of the encrypted password;
receiving (S16) an access request which is sent by the user device in order to request the user access, the access request comprising a decrypted password;
verifying (S17) whether the decrypted password matches the password to be encrypted;
if the decrypted password matches the password to be encrypted:
granting (S18) the user access.

2. The method (M1) of claim 1,
wherein the encrypted password is generated in an asymmetric or hybrid encryption method by using a public key, which is stored in a memory of the medical device, the public key forming a key pair with a private key for generating (S33) the decrypted password.

3. The method (M1) of claim 1, further comprising:
generating (S11) the password to be encrypted.

4. The method (M1) of claim 3, further comprising:
starting (S12) a timer in response to the generation (S11) of the password to be encrypted;
when the timer has elapsed:
generating (S13) a new version of the password to be encrypted;
overwriting (S13) a current version of the password to be encrypted with the new version.

5, The method (M1) of claim 3,
wherein the password to be encrypted is newly generated each time the medical device is switched on or off or switches between different operating modes for the operation of the medical device; and/or
wherein the password to be encrypted is generated by using a random generator for generating a random character string.

6. The method (M1) of claim 1,
wherein the access request further comprises a user ID, which is uniquely assigned to a user of the user device;
wherein whether the user has access authorization is verified with the aid of the user ID;
wherein the user access is granted only if the user has access authorization.

7. A method (M2) for requesting user access to a medical device via a user device, wherein the method (M2) is carried out by a processor of the user device and comprises:
receiving (S21) an encrypted password sent by the medical device;
generating (S22) a password request, which comprises the encrypted password, for requesting a decrypted password;
sending (S23) the password request to a decryption device for decrypting the encrypted password;
receiving (S24) a decrypted password sent by the decryption device;
generating (S25) an access request, which comprises the decrypted password, for requesting the user access;
sending (S26) the access request to the medical device.

8. A method (M3) for operating a decryption device, wherein the method is carried out by a processor of the decryption device and comprises:
receiving (S31) a password request, which is sent by a user device, for requesting a decrypted password, the password request comprising an encrypted password, the encrypted password having been generated by a processor of a medical device by encrypting a password to be encrypted;
generating (S33) the decrypted password by decrypting the encrypted password;
sending (S34) the decrypted password to the user device.

9. The method (M3) of claim 8,
wherein the password request further comprises a user ID, which is uniquely assigned to a user of the user device;
wherein whether the user has access authorization is verified with the aid of the user ID;
wherein the decrypted password is generated and/or sent to the user device only if the user has access authorization.

10. The method (M3) of claim 8,
wherein the decrypted password is generated by using a private key, which is stored in a memory of the decryption device, the private key forming a key pair with a public key for generating (S14) the encrypted password in an asymmetric or hybrid encryption method.

11. A method (M) for controlling user access to a medical device via a user device, wherein the method (M) comprises:
steps (S11, S12, S13, S14, S15, S16, S17, S18, S19) of the method (M1) as set forth in claim 1.

12. A method (M) for controlling user access to a medical device via a user device, wherein the method (M) comprises:
steps (S21, S22, S23, S24, S25, S26) of the method (M2) as set forth in claim 7.

13. A method (M) for controlling user access to a medical device via a user device, wherein the method (M) comprises:
steps (S31, S32, S33, S34, S35) of the method (M3) as set forth in claim 8.

14. An apparatus for data processing, comprising:
a first processor, which is configured to carry out the method (M1) as set forth in claim 1.

15. An apparatus for data processing, comprising:
a second processor, which is configured to carry out the method (M2) as set forth in claim 7.

16. An apparatus for data processing, comprising:
a third processor, which is configured to carry out the method (M3) as set forth in claim 8.
